(19) United States
(12) Patent Application Publication
(10) Pub. No.: US 2016/0331708 A1
(43) Pub. Date: Nov. 17, 2016

(54) MEANS AND METHODS FOR INCREASING ANTIBIOTIC ACTIVITY

(71) Applicant: UNIVERSITEIT LEIDEN, Leiden (NL)

(72) Inventors: Hye Kyong Kim, Leiden (NL); Anne van der Meij, Leiden (NL); Robert Verpoorte, Leiden (NL); Young Hae Choi, Leiden (NL); Gilles Philippus van Wezel, Leiden (NL)

(21) Appl. No.: 15/110,936

(22) PCT Filed: Jan. 9, 2015
(86) PCT No.: PCT/NL2015/050015

§ 371 Date: Jul. 11, 2016

Related U.S. Application Data
(60) Provisional application No. 61/925,303, filed on Jan. 9, 2014.

(30) Foreign Application Priority Data
Jan. 9, 2014 (EP) 14150644.4

(51) Int. Cl. A61K 31/196; A61K 31/7036; A61K 31/165; A61K 31/7056; A61K 31/43; A61K 31/545; A61K 38/14; A61K 38/08; A61K 31/496; C12P 19/54; C12P 19/52; C12P 19/48; C12P 13/02; C12P 37/00; A61K 35/74

(52) U.S. Cl. CPC A61K 31/196; A61K 35/74; A61K 31/7036; A61K 31/165; A61K 31/7056; A61K 31/43; A61K 31/545; A61K 38/14; A61K 38/08; A61K 31/496; C12P 19/54; C12P 19/52; C12P 19/485; C12P 13/02; C12P 37/00

(57) ABSTRACT

The disclosure provides means and methods for increasing the activity of antibiotics. This enables the use of lower antibiotic dosages and the treatment of multidrug-resistant bacteria.

### CROSS-REFERENCE TO RELATED APPLICATIONS

This application is a national phase entry under 35 U.S.C. §371 of International Patent Application PCT/NL2015/050015, filed Jan. 9, 2015, designating the United States of America and published in English as International Patent Publication WO 2015/105423 A1 on Jul. 16, 2015, which claims the benefit under Article 8 of the Patent Cooperation Treaty to European Patent Application Serial No. 14150644.4, filed Jan. 9, 2014, and under 35 U.S.C. §199 to U.S. Provisional Patent Application Ser. No. 61/925,303, also filed on Jan. 9, 2014.

### BACKGROUND

Antibiotics have been used for decades in order to combat bacterial and fungal infections. Numerous antibiotics were originally primarily obtained from soil microorganisms, which led to the near eradication of diseases such as tuberculosis (Hopwood, 2007). Non-limiting examples of commonly used antibiotics are penicillins, cephalosporins, aminoglycosides and glycopeptides antibiotics. Nowadays, antibiotics that are semisynthetic modifications of natural compounds are also used.

The emergence of infectious diseases involving multidrug-resistant (MDR) bacterial pathogens since the 1980s means that bacterial infections are still a major threat for human health. According to the World Health Organization (WHO), around 440,000 new cases of multidrug-resistant tuberculosis (MDR-TB) are found annually, causing more than 150,000 deaths. Extensively drug-resistant tuberculosis (XDR-TB) has now been reported in 64 countries to date (WHO-Media-centre, 2012). The explosive increase in infections by pathogens such as methicillin-resistant Staphylococcus aureus (MRSA), vancomycin-resistant Enterococcus faecium (VRE) and fluoroquinolone-resistant Pseudomonas aeruginosa is estimated to cause approximately 19,000 deaths per year in the U.S. (Klevens et al., 2007), and the most recent occurrence of panantibiotic-resistant infections pose the grave threat of completely untreatable infections (Arias and Murray, 2009).

Antibiotic-resistant bacterial strains, for instance, emerge after prolonged antibiotic treatment, resulting in one or more mutations in the bacterial genome or the acquisition of extra-chromosomal DNA. As a result, a bacterial strain may become resistant to the antibiotic, meaning that it can endure high concentrations of the antibiotic, so that even higher doses would be required in order to counteract the resistant strain. However, a higher antibiotic dose involves an increased chance of adverse side-effects and is, therefore, not always a feasible option.

### BRIEF SUMMARY

Provided are methods for increasing the antibacterial activity of antibiotics, so that therapeutic doses can be lowered. Further provided are means and methods for counteracting antibiotic-resistant bacteria.

The disclosure provides the surprising insight that the antibacterial activity of antibiotics is increased by anthranilic acid. Alternatively, a pharmaceutically acceptable salt, ester, hydrate, solvate or functional derivative of anthranilic acid may be used. Although anthranilic acid itself does not show much antibacterial activity, unless used in very high concentrations, anthranilic acid or anthranilic acid-based compounds appear to reduce the minimum inhibitory concentration (MIC) of antibiotics. This means that a lower amount of antibiotic is required to inhibit the growth of bacteria. An important implication of this insight of this disclosure is the fact that (the risk of) adverse side effects of antibiotics, such as, for instance, nausea, headache, diarrhea, fever, allergic reactions and even anaphylactic shock, can now be reduced because lower antibiotic dosages are needed when the antibiotic is used in combination with anthranilic acid or a pharmaceutically acceptable salt, ester, hydrate, solvate or functional derivative thereof. Furthermore, treatment of antibiotic-resistant bacterial strains and multidrug-resistant strains has now become possible. While high antibiotic concentrations would be required before this disclosure to treat such resistant bacteria, which is in practice not feasible, now it has become possible to treat such resistant bacteria with more acceptable antibiotic dosages due to the synergistic effect of anthranilic acid and pharmaceutically acceptable salts, esters, hydrates, solvates and functional derivatives thereof. This enables the reuse of antibiotics that have nowadays been abandoned due to resistance problems. Of note, anthranilic acid is a food grade compound, with a proven safety profile for humans, which facilitates therapeutic applications in humans.

Accordingly, this disclosure provides an antibiotic and anthranilic acid for use in the treatment of a subject suffering from or at risk of suffering from a bacterial infection. Alternatively, a pharmaceutically acceptable salt, ester, hydrate, solvate or functional derivative of anthranilic acid is used.

Further provided is, therefore, an antibiotic and a pharmaceutically acceptable salt, ester, hydrate, solvate or functional derivative of anthranilic acid for use in the treatment of a subject suffering from or at risk of suffering from a bacterial infection. Also provided is a use of an antibiotic and anthranilic acid in the preparation of a medicament for preventing or treating a bacterial infection. A use of an antibiotic and a pharmaceutically acceptable salt, ester, hydrate, solvate or functional derivative of anthranilic acid in the preparation of a medicament for preventing or treating a bacterial infection is also provided herewith.

Anthranilic acid, also named 2-aminobenzoic acid, ortho-aminobenzoic acid or vitamin L1, is an aromatic acid with the formula shown in FIG. 2.

It is typically formed as an intermediate product during the production of saccharin and azo dyes, and it is used for preparing perfumes imitating orange and jasmine. As stated above, anthranilic acid is a food grade compound so that it can be safely used for humans. Its use for increasing the antibacterial activity of antibiotics has not been disclosed before.

As used herein, a salt of anthranilic acid means a compound wherein the carboxylic group of anthranilic acid is deprotonated, so that its conjugated base forms a carboxylate anion (“anthranilate”), which may, for instance, be present as an anion in solution or be complexed with a cation.

An ester of anthranilic acid is a product formed when anthranilic acid is reacted with a hydroxyl compound such as, for instance, an alcohol.

A solvate of anthranilic acid is an anthranilic acid-based ion which is complexed by solvent molecules.

A functional derivative of anthranilic acid is an anthranilic acid compound which has been altered such that it retains the same capability of increasing the antibacterial activity of an antibiotic as anthranilic acid (in kind, not necessarily in amount). A functional derivative of anthranilic acid is typically formed by substituting one or more non-essential groups of anthranilic acid, such as, for instance, one or more hydrogen atoms. Preferred functional derivatives are indicated in table 1. A preferred functional derivative is an anthranilic acid wherein one or more non-essential hydrogen atoms is replaced by hydrogen, Br, Cl, I, or (CH₂)ₙ—CH₃ wherein n=0, 1, 2 or 3, while typically maintaining the carboxyl group, as well as the amino group at the ortho position. As used herein, a pharmaceutically acceptable salt, ester, hydrate, solvate or functional derivative of anthranilic acid is also referred to as “an anthranilic acid-based compound.”

The term “pharmaceutically acceptable” means that a compound is suitable for therapeutic use in humans.

The term “antibiotic” is defined herein as a compound, produced by or derived from a (micro)organism, that can counteract the growth of other microorganisms, in particular bacteria. Also, synthetic variants of antibacterial compounds originally derived from (micro)organisms are embraced by the term antibiotic. Antibiotics are typically classified based on their mechanism of action, chemical structure, or spectrum of activity. Most antibiotics interfere with bacterial integrity or function. Non-limiting examples of antibiotics that target the bacterial cell wall are penicillins and cephalosporins, while polymyxins disrupt the cell membrane. Antibiotics like rifamycins, lipiarmycins, quinolones and sulfonamides interfere with essential bacterial enzymes, whereas macrolides, lincosamides and tetracyclines interfere with protein synthesis. Further categorization of antibiotics is based on their target specificity. “Narrow-spectrum” antibiotics target specific types of bacteria, such as Gram-negative or Gram-positive bacteria, whereas broad-spectrum antibiotics affect a wide range of bacteria. As used herein, the term antibiotic embraces bactericidal antibiotics, which kill bacteria, as well as bacteriostatic antibiotics, which inhibit bacterial reproduction.

As used herein, a referral to “an antibiotic” means “one or more antibiotics.” Hence, a singular term embraces a plural term, and vice versa.

An “antibiotic-resistant” bacterial strain as referred to herein means a bacterial strain which has a reduced sensitivity for at least one antibiotic as compared to other, non-resistant bacteria. This means that commonly used doses of an antibiotic are not capable of inactivating all bacteria of such strain. Often, antibiotic-resistant strains could in theory be killed or inactivated using extremely high doses of antibiotic, but since such high doses are not tolerated in humans, treatment has become problematic.

Multidrug-resistant bacteria are defined herein as bacteria which have a reduced sensitivity for at least two, but preferably at least three, four, five or ten different drugs, preferably for at least two, or at least three, four, five or ten, different kinds of antibiotics. Such multidrug-resistant bacteria were particularly problematic before this disclosure.

Now that the disclosure has provided the insight that a combination of anthranilic acid, or an anthranilic acid-based compound, with an antibiotic has an increased antibacterial activity as compared to antibiotics in the absence of anthranilic acid or anthranilic acid-based compounds, effective methods for counteracting bacterial activity and/or growth are provided. Preferably, a combination of an antibiotic and anthranilic acid (or an anthranilic acid-based compound) is used for killing bacteria, meaning that the structure and/or function of the bacteria becomes so disrupted that the bacteria are no longer viable. As explained before, an important advantage of a method according to this disclosure is the fact that a lower dose of antibiotic can be used. Alternatively, with the same dose of antibiotics, a higher bactericidal or bacteriostatic effect is obtained, which, for instance, allows counteracting antibiotic-resistant bacteria. One aspect of the disclosure, therefore, provides a method for counteracting bacterial activity and/or growth, comprising exposing bacteria to:
an antibiotic; and
anthranilic acid, or a pharmaceutically acceptable salt, ester, hydrate, solvate or functional derivative thereof.

Further provided is a use of anthranilic acid, or a pharmaceutically acceptable salt, ester, hydrate, solvate or functional derivative thereof, for increasing the antibacterial activity of an antibiotic. A preferred embodiment provides a use of anthranilic acid, or a pharmaceutically acceptable salt, ester, hydrate, solvate or functional derivative thereof, for decreasing the minimum inhibitory concentration (MIC) of an antibiotic. The MIC of an antibiotic is the lowest concentration of the antibiotic that inhibits the growth of a microorganism overnight. This is preferably measured using the MIC test as described in the examples. For instance, as shown in the Examples, Streptomycin has a MIC value of 31.2 μg/ml for Bacillus subtilis. When anthranilic acid or an anthranilic acid-based compound is present in the culture, this MIC value of Streptomycin is reduced. For instance, if anthranilic acid is present in the culture in a concentration of about 50 μg/ml, the MIC value of Streptomycin is reduced by a factor 2. If anthranilic acid is present in a concentration of about 150 μg/ml, the MIC value of Streptomycin is reduced by a factor 3 and if anthranilic acid is present in a concentration of about 300 μg/ml, the MIC value of Streptomycin is even reduced by a factor 6. So, anthranilic acid and anthranilic acid-based compounds are capable of considerably reducing the MIC value of an antibiotic and thus enhancing the efficacy of the antibiotic against pathogenic bacteria. One preferred embodiment, therefore, provides a use of anthranilic acid, or a pharmaceutically acceptable salt, ester, hydrate, solvate or functional derivative thereof, for increasing the antibacterial activity of an antibiotic, wherein the MIC of the antibiotic is decreased with at least a factor 2. This means that the lowest concentration of the antibiotic that inhibits the growth of a microorganism overnight is two times lower in the presence of anthranilic acid or an anthranilic acid-based compound, as compared to a situation wherein anthranilic acid, or an anthranilic acid-based compound, is not present. Another preferred embodiment provides a use of anthranilic acid, or a pharmaceutically acceptable salt, ester, hydrate, solvate or functional derivative thereof, for increasing the antibacterial activity of an antibiotic, wherein the MIC of the antibiotic is decreased with at least a factor 4. Even more preferably, the MIC is decreased with at least a factor 6.

One important application of this disclosure is in medicine. A subject suffering from, or at risk of suffering from, a bacterial infection can be provided with an antibiotic and with anthranilic acid or an anthranilic acid-based compound, in order to prevent or treat the infection. One preferred embodiment thus provides a method for treating a subject suffering from, or at risk of suffering from, a bacterial infection, the method comprising administering to the subject:
an antibiotic; and
anthranilic acid, or a pharmaceutically acceptable salt, ester, hydrate, solvate or functional derivative thereof.

As used herein, a combination of an antibiotic with anthranilic acid, or with an anthranilic acid-based compound, is referred to as “a combination according to the disclosure.” In one embodiment, antibiotic and anthranilic acid (or an anthranilic acid-based compound) are administered to a subject simultaneously. Alternatively, the antibiotic and anthranilic acid (or anthranilic acid-based compound) are administered consecutively. For instance, in one embodiment, an antibiotic is firstly administered to a subject, where after anthranilic acid or an anthranilic acid-based compound is administered. In another embodiment, anthranilic acid or an anthranilic acid-based compound is firstly administered to a subject, where after an antibiotic is administered. All these above-mentioned embodiments are embraced within the term “combination according to this disclosure.” Preferably, if an antibiotic and anthranilic acid or an anthranilic acid-based compound are administered consecutively, they are both administered within 12 hours, preferably within 10 hours, more preferably within 8, 6, 4 or 2 hours, so that their synergistic therapeutic activity is optimal. In one preferred embodiment, an antibiotic and anthranilic acid or an anthranilic acid-based compound are administered to a subject within one hour.

The antibiotic and anthranilic acid or anthranilic acid-based compound are typically administered to a subject in a therapeutically effective amount, meaning that the dosages are sufficient to combat a bacterial infection.

The amounts of antibiotic and anthranilic acid or anthranilic acid-based compound to be administered to a subject should typically be in the therapeutic window, meaning that sufficient amounts of the antibiotic and the anthranilic acid (-based compound) are used for obtaining a therapeutic effect, while the amounts do not exceed a threshold value leading to an unacceptable extent of side-effects. The lower the amount of antibiotic needed for obtaining a desired therapeutic effect, the larger the therapeutic window will typically be. A combination of an antibiotic with anthranilic acid, or with an anthranilic acid-based compound, will typically enlarge the therapeutic window and is, therefore, advantageous.

Dose ranges of combinations according to the disclosure to be used in the therapeutic applications as described herein are typically designed on the basis of rising dose studies in the clinic in clinical trials for which rigorous protocol requirements exist.

Preferably, anthranilic acid is used for humans in a concentration of 1-500 mg/kg body weight.

Antibiotics for use according to this disclosure are preferably selected from the group consisting of antibiotics that inhibit bacterial protein synthesis, antibiotics that inhibit cell wall synthesis, antibiotics that disrupt peptidoglycan cross-linkage, antibiotics that inhibit bacterial DNA synthesis and antibiotics that inhibit bacterial RNA synthesis. As shown in the Examples, the antibacterial activity of different members of each of the above mentioned groups is increased by anthranilic acid (or an anthranilic acid-based compound). In a particularly preferred embodiment, an antibiotic is used that is selected from the group consisting of streptomycin, neomycin, kanamycin, gentamycin, clindamycin, penicillin G, cephalexin, actinomycin C3, and pharmaceutically acceptable salts, esters, hydrates and solvates thereof.

In one preferred embodiment, a combination of an antibiotic and anthranilic acid (or an anthranilic acid-based compound) according to the disclosure is used wherein the dosage of the antibiotic is significantly lower as compared to conventional doses which have been used before this disclosure. Preferably, an antibiotic dose is used that is at most half the amount that would be required for killing bacteria in the absence of anthranilic acid (or an anthranilic acid-based compound). According to this disclosure, the use of such low doses has become possible due to the synergistic effect of anthranilic acid and anthranilic acid-based compounds. Further provided is, therefore, an antibiotic and anthranilic acid (or an anthranilic acid-based compound) for use according to the disclosure, or a method or use according to the disclosure, wherein an amount of the antibiotic is used that is at most half the amount required for killing bacteria in the absence of anthranilic acid (or an anthranilic acid-based compound). Preferably, an antibiotic dose is used that is at most one third, preferably at most one fourth, one fifth or one sixth, of the amount required for killing bacteria in the absence of anthranilic acid (or an anthranilic acid-based compound).

In one preferred embodiment, streptomycin (sulfate) is administered to a subject in an amount of less than 25 mg/kg, and/or neomycin (sulfate) is administered to a subject in an amount of less than 15 mg/kg, and/or kanamycin (sulfate) is administered to a subject in an amount of less than 15 mg/kg, and/or gentamycin (sulfate) is administered to a subject in an amount of less than 15 mg/kg, and/or clindamycin (HCL) is administered to a subject in an amount of less than 25 mg/kg, and/or penicillin G (sodium salt) is administered to a subject in an amount of less than 300.000 units/kg, and/or cephalexin (1H₂O) is administered to a subject in an amount of less than 15 mg/kg.

In another preferred embodiment, a combination of an antibiotic and anthranilic acid (or an anthranilic acid-based compound) according to the disclosure is used against antibiotic-resistant bacteria. This allows reuse of antibiotics that have nowadays been abandoned due to resistance problems. In a particularly preferred embodiment, the combination of an antibiotic and anthranilic acid (or an anthranilic acid-based compound) is used against multidrug-resistant bacteria.

In another preferred embodiment, a combination of an antibiotic and anthranilic acid (or an anthranilic acid-based compound) is used against Acinetobacter baumanii, Clostridium difficile, Enterobacter cloacae, Escherichia coli, Klebsiella pneumoniae, Mycobacterium tuberculosis, Neisseria gonorrhoeae, Pseudomonas aeruginosa, Staphylococcus aureus and Salmonella typhimurium.

Subjects that can be treated with a method according to this disclosure encompass mammals such as human subjects, cats, dogs, apes, monkeys, horses, rabbits and cattle like cows, sheep and goats. In a preferred embodiment, the subject is a human individual. Preferably, the individual is a child with an age below 18 years or an elderly person with an age above 50 years or, preferably, above 60 years. These humans are typically at a higher risk of adverse side-effects of antibiotics, so that a lower antibiotic dose, which has become possible due to the effect of anthranilic acid or an anthranilic acid-based compound, is particularly advantageous for children and elderly. In yet another embodiment, the subject is an immune-compromised human, such as, for instance, a human individual who has received chemotherapy or radiation therapy, or an individual suffering from HIV infection/AIDS. These individuals also typically have a higher risk of adverse side-effects of antibiotics. A method according to the disclosure is, therefore, also particularly advantageous for this specific group of individuals.

If a subject is infected with an antibiotic-resistant bacterial strain, or multidrug-resistant strain, treatment would be very difficult before this disclosure because too high doses of antibiotics are not tolerated. Now that the disclosure has provided a way for increasing antibacterial activity of antibiotics, antibiotic treatment of certain antibiotic-resistant bacterial strains has become possible again, using acceptable antibiotic doses in combination with anthranilic acid or an anthranilic acid-based compound. This is particularly advantageous for children with an age below 18 years, elderly persons with an age above 50 or 60 years, and immune-compromised individuals as described above.

Even though the methods according to this disclosure allow the lowering of antibiotic doses, treatment should still be given with care. A test whether a subject is actually suffering from an infection by pathogenic bacteria is often preferred before treatment is started. Further provided is, therefore, a method for treating a subject suffering from a bacterial infection, the method comprising:
measuring whether a sample from the subject comprises pathogenic bacteria, proteins from pathogenic bacteria, nucleic acid from pathogenic bacteria, or antibodies against pathogenic bacteria; and
administering to the subject an antibiotic and anthranilic acid, or an antibiotic and a pharmaceutically acceptable salt, ester, hydrate, solvate or functional derivative of anthranilic acid, if the sample comprises pathogenic bacteria or proteins from pathogenic bacteria or nucleic acid from pathogenic bacteria or antibodies against pathogenic bacteria.

By measuring the presence of pathogenic bacteria or protein or nucleic acid thereof, or antibodies against such bacteria, in a sample it is established that treatment is indeed required. In a preferred embodiment, the pathogenic bacterium is a bacterium selected from Acinetobacter baumanii, Clostridium difficile, Enterobacter cloacae, Escherichia coli, Klebsiella pneumoniae, Mycobacterium tuberculosis, Neisseria gonorrhoeae, Pseudomonas aeruginosa, Staphylococcus aureus and Salmonella typhimurium.

In one embodiment, it is measured whether a sample from the subject comprises antibiotic-resistant bacteria (or multidrug-resistant bacteria), or protein or nucleic acid from such resistant bacteria, or antibodies against such resistant bacteria. Before this disclosure, if infection of antibiotic-resistant or multidrug-resistant bacteria is established, treatment would be problematic. With a method according to this disclosure, however, treatment can be started with acceptable antibiotic doses, due to the effect of anthranilic acid or an anthranilic acid-based compound.

It is not always possible or desirable to perform sample testing before treatment, for instance, when direct start of therapy is preferred in view of clinical symptoms or in situations where proper test equipment is unavailable. In such cases, treatment of subjects suffering from, or at risk of suffering from, a bacterial infection can be directly started, using a combination according to this disclosure.

This disclosure further provides a composition or kit of parts, comprising a combination according to this disclosure. As explained herein before, preferably half, or less, of the conventional antibiotic doses are used, so that (the risks of) adverse side effects are decreased. The co-administration of anthranilic acid or an anthranilic acid-based compound (either simultaneously or consecutively) enables the use of such low antibiotic dosages. A composition or kit of parts comprising unit doses which contain such low dosages of antibiotic is, therefore, preferred. Further provided is, therefore, a composition or kit of parts, comprising:
anthranilic acid, or a pharmaceutically acceptable salt, ester, hydrate, solvate or functional derivative thereof; and
an antibiotic in a unit dose that is at most half the amount required for killing bacteria in the absence of anthranilic acid or an anthranilic acid-based compound.

As discussed above, the antibiotic is preferably selected from the group consisting of antibiotics that inhibit bacterial protein synthesis, antibiotics that inhibit cell wall synthesis, antibiotics that disrupt peptidoglycan cross-linkage, antibiotics that inhibit bacterial DNA synthesis and antibiotics that inhibit bacterial RNA synthesis. Most preferably, the antibiotic is selected from the group consisting of streptomycin, neomycin, kanamycin, gentamycin, clindamycin, penicillin G, cephalexin, actinomycin C3, and pharmaceutically acceptable salts, esters, hydrates and solvates thereof. The use of significantly lower doses of these antibiotics is particularly preferred in order to reduce (the risks of) side effects. Further provided is, therefore, a composition or kit of parts according to the disclosure, wherein streptomycin (sulfate) is present in at least one unit dose of less than 1.5 gram active compound, and/or wherein neomycin (sulfate) is present in at least one unit dose of less than 1 gram active compound, and/or wherein kanamycin (sulfate) is present in at least one unit dose of less than 1 gram active compound, and/or wherein gentamycin (sulfate) is present in at least one unit dose of less than 1 gram active compound, and/or wherein clindamycin (HCL) is present in at least one unit dose of less than 1.5 gram active compound, and/or wherein penicillin G (sodium salt) is present in at least one unit dose of less than 20×10⁶ units active compound, and/or wherein cephalexin (1H₂O) is present in at least one unit dose of less than 1 gram active compound.

A composition according to the disclosure is preferably a pharmaceutical composition. Such pharmaceutical composition typically comprises a pharmaceutically acceptable carrier, diluent or excipient such as, for instance, saline or fatty oil. Examples of carriers and excipients which can be incorporated in tablets, capsules and the like are a binder such as gum tragacanth, acacia, corn starch or gelatin; microcrystalline cellulose; corn starch, pregelatinized starch, alginic acid and the like; or a lubricant such as magnesium stearate. A pharmaceutical composition according to the disclosure is preferably suitable for human use. Non-limiting examples of pharmaceutical compositions according to this disclosure are tablets, capsules, syrups, elixirs, suppositories and injection solutions. A tablet or capsule is preferred in view of easy administration and storage.

A composition or kit of parts according to the disclosure, as described above, is particularly suitable for administration to bacteria in order to counteract bacterial growth, preferably in order to kill the bacteria. Furthermore, a composition or kit of parts according to the disclosure is particularly suitable for administration to a subject in order to prevent or treat a bacterial infection. Further provided is, therefore, a method for counteracting bacterial activity and/or growth, and/or for treating a subject suffering from, or at risk of suffering from, a bacterial infection, the method comprising administering a composition or kit of parts according to the disclosure to the bacteria or to the subject.

A composition or kit of parts according to the disclosure preferably comprises an antibiotic for use against antibiotic-resistant bacteria and/or multidrug-resistant bacteria.

In this disclosure it was found that micro-organisms can be responsive to plant-derived compounds. Such micro-organisms adapt their metabolism in response to the presence of such plant-derived compounds in their environment. As a result compounds are produced that are not, or much less produced when the plant-derived compound is not present. The disclosure utilizes this finding among others in the identification of compounds with antimicrobial activity. The disclosure, therefore, also provides a method for culturing a micro-organism comprising culturing the micro-organism in the presence of a plant-derived compound and determining whether the micro-organism produces a compound with antimicrobial activity and/or a compound that reduces the resistance of bacteria to an antibiotic. The plant-derived compound is preferably a plant hormone. A compound is said to have antimicrobial activity when, at a given concentration, it inhibits growth of a microbe but not essentially the growth of an animal or plant cell. A compound is also said to have antimicrobial activity when it, in combination with an antibiotic, at a given concentration, inhibits growth of a microbe but not essentially the growth of an animal or plant cell, and that inhibition is greater than the inhibition of growth by the antibiotic itself (of course in the absence of the compound and tested under otherwise similar conditions). The latter compound is also referred to herein as a compound that reduces the resistance of bacteria to an antibiotic. The test for antibiotic activity on a microbe is preferably done using the MIC assay as described in the examples. The compound can thus have antimicrobial activity on its own or in combination with an antibiotic. The microbe is preferably a pathogenic bacterium, preferably as defined herein. Without being bound by theory it is believed that plants interact with the micro-organism flora that surrounds them and influence, among others, the composition of the micro-organism flora in their direct vicinity. In this disclosure, it was found that micro-organisms are responsive to plant hormones and respond to the presence by altering the metabolism of compounds with antimicrobial activity. This disclosure utilizes the responsiveness to plant hormones among others, to identify compounds that either alone or in combination with an antibiotic, exhibit antimicrobial activity. The terms antibiotic and compound with antimicrobial activity are used interchangeably herein.

The plant hormone is preferably an auxin, ethylene, a jasmonate or an OPDA (oxyphytodienoic acid). The plant hormone is preferably a jasmonate or 10-OPDA or 12-OPDA. Various jasmonates and OPDA and their biosynthesis are described in Creelman and Mullet (1997; Annu. Rev. Plant Physiol. Plant Mol. Biol. 1997, 48:355-81) and Wasternack (2007; Ann. Bot. 2007 October; 100(4):681-697), which are incorporated by reference herein. The plant hormone is preferably a jasmonate. Preferred jasmonates are jasmonic acid, methyl jasmonate and jasmone. The jasmonate is preferably methyl jasmonate. The micro-organism in a method for culturing as indicated above is preferably an Actinomycete. The Actinomycete is preferably of the genus Streptomyces.

The disclosure is further explained in the following examples. These examples do not limit the scope of the invention, but merely serve to clarify the invention.

### BRIEF DESCRIPTION OF THE DRAWINGS

FIG. 1. Effect of methyl jasmonate and salicylic acid on the antimicrobial activity of Streptomyces sp MBT3. Top, TLC biogram of EtOAc extracts of the culture fluid of MBT3. Cultures were grown in the presence of (1) control (water); (2), ethylacetate; (3), methyl jasmonate; (4), Salicylic acid. EtOAc extracts were separated on a TLC plate and the TLC plate was then assayed for antimicrobial activity using an overlay assay with B. subtilis as the indicator strain, which was incubated O/N at 37° C. to assess the inhibition zones. TLC solvent was a 10:1 mixture of chloroform and methanol. Antimicrobial activity is visible as a zone of clearance. Bottom, corresponding ¹H NMR spectra of the extracts.

FIG. 2. Chemical structure of anthranilic acid

FIG. 3. Effect of anthranilic acid on the MIC value of streptomycin. The MIC is reduced from 30 μg/ml (no anthranilic acid) to 5 μg/ml (at 300 μg/ml anthranilic acid).

### DETAILED DESCRIPTION

### Examples

### Materials & Methods

### Preparation of Chemical Solutions

Anthranilic acid and the structurally related catechol, benzoic acid, 2,3-dihydroxybenzoic acid, salicylic acid, L-ascorbic acid, L-proline, D,L-proline, 3,5-diaminobenzoic acid, 3-aminobenzoic acid, 4-aminobenzoic acid and anthranilic acid amide were dissolved in water and added from a concentrated stock solution (1.5-10 mg/ml in distilled water, depending on the compound). All antibiotics were dissolved in water except actinomycin and rifampin which were dissolved in 5% aqueous ethanol and 10% DMSO, respectively.

### Growth of Streptomyces and Preparation of Extracts

Streptomyces MBT3 was obtained from forest soil in the Netherlands (Zhu, 2014).

All media for growth of Streptomyces are described in (Kieser et al., 2000). For the preparation of Streptomyces cultures, spores were obtained from SFM agar plates and inoculated at a density of 5×10⁶ into 20 ml liquid minimal media with mannitol and glycerol as the carbon sources culture media in a 250 ml Erlenmeyer flask and incubated at 30° C. in a rotary shaker (250 rpm). Mycelia were precipitated by centrifugation for 10 minutes at 12,000 g. Each replicate of culture supernatant was extracted twice with an equal amount (40 ml in total) of analytically pure EtOAc (Sigma-Aldrich St. Louis, Mo., USA). Two grams of Na₂SO₄ was then added into the organic phase to remove remaining water and was evaporated under vacuum at 38° C. Samples were then re-dissolved in Methanol-d₄ (99.8%) (Cambridge Isotope Laboratories, Inc., Andover, Mass., USA) for NMR analysis. 1.5 ml of the culture was collected every three hours and centrifuged. Supernatants were separated from the pellet fraction and used to assess antimicrobial activity, while the pellet dry weight was used for biomass determination (three replicates for each time point) following heating at 70° C. overnight. The growth as indicated by dried biomass was plotted against time.

### Antibiotic MIC Tests in Microtiter Plates

Bacillus subtilis 168 was grown on LB agar plates (Sambrook et al., 1989) and a single colony was used for inoculation of a liquid LB culture. This was culture was grown to an OD₆₀₀ of around 0.5 (giving a density of 10⁷-10⁸ cfu/ml). The minimal inhibitory concentration (MIC) was determined according to the guidelines described by the BSAC (British Society of Antimicrobial Chemotherapy), using 96-well microtiter plates (MTP). First, 100 μl of LB broth with indicator strain was added to each well. 100 μl of a test solution (compound or control solvent and with or without added anthranilate) was added to the first well and mixed with the culture broth. From this suspension of in total 200 μl, 100 μl was transferred to the next well, mixed, transferred to the third well and so on, so as to give a two-fold dilution series of the compound mixture. As the negative control, solvent (water, 5% EtOH or 10% DMSO) was used. 96-well plates were incubated at 37° C. overnight and bacterial growth determined visually and using a MTP plate reader.

### Thin Layer Chromatography (TLC) and Agar Overlay Antimicrobial Assay

TLC silica gel 60 F₂₅₄ (Merck, Darmstatd, Germany) plates were developed using chloroform (CHCl₃) and methanol (MeOH) as the solvent system and visualized under UV light 254 nm and 365 nm. TLC-bioautography assays were done by placing the developed TLC plate onto a bioassay petri dish overlaid with soft LB agar (Hispanagar) (0.6%) containing Bacillus subtilis as an indicator. Following two hour incubation, the TLC plate was removed and incubated overnight at 37° C. A control plate was also processed using the same solvents excluding test material to ensure the TLC and solvents themselves do not affect the growth of the indicator strain. The activity assessment was based on inhibition zones of the indicator strain.

### Mass Spectrometry

Fourier Transform Mass Spectrometry (FTMS) (Bruker) was conducted on the purified compounds to determine the exact mass of the compounds. The analyses were performed by DI-nanoESI-MS in the positive ion mode using the automated Advion NanoMate Triversa system (type “A” chip) coupled to a LTQ-FT Ultra (Thermo Fisher Scientific). Mass spectra were recorded using three scan ranges containing 20 scans: 50-250; 250-500; 500-1,000 m/z (in this order) at a resolution of 100,000. Separate scan ranges instead of one full scan range was chosen in order to enhance sensitivity. The MS was tuned with inlet capillary temperature of 120° C., capillary voltage of 35 V and the tube lens voltage of 50 V.

### NMR Measurement of Whole Supernatant Extract

¹H NMR spectra were recorded at 25° C. on a 500 MHz Bruker DMX-500 spectrometer (Bruker, Karlsruhe, Germany) operating at a proton NMR frequency of 500.13 MHz. Methanol-d4 was used as the internal lock. Each ¹H NMR spectrum consisted of 128 scans requiring 10 minutes and 26 seconds acquisition time with the following parameters: 0.16 Hz/point, pulse width (PW)=30° (11.3 μsec), and relaxation delay (RD)=1.5 sec. A pre-saturation sequence was used to suppress the residual H₂O signal with low power selective irradiation at the H₂O frequency during the recycle delay. FIDs were Fourier transformed with LB=0.3 Hz. The resulting spectra were manually phased and baseline corrected, and calibrated to MeOH-d4 at 3.3 ppm, using XW1N NMR (version 3.5, Bruker). 2D NMR techniques were performed on a 600 MHz Bruker DMX-600 spectrometer (Bruker, Karlsruhe, Germany) operating at a proton NMR frequency of 600.13 MHz. Detailed NMR parameters were used as previously described (Kim et al., 2010).

### Data Processing and Multivariate Analysis

¹H NMR spectra were manually phased, baseline corrected and calibrated. ¹H-NMR spectra were further automatically converted to ASCII files using AMIX (v. 3.7, Bruker Biospin). Spectral intensities were scaled to total intensity and the region of δ 0.3-10.0 was reduced to integrated regions of width (0.04 ppm). The regions δ 4.7-5.0 and δ 3.30-3.34 were excluded from the analysis because of the residual signal of H₂O and methanol-d4, respectively. Data were extracted after acquiring the spectra by “binning” or “bucketing,” in which spectra are split into discrete regions and integrated (Jellema, 2009). Principal component analysis (PCA) with scaling based on Pareto while Partial Least Square-Discriminant Analysis (PLS-DA) and Orthogonal Projection to Latent Structures (OPLS) were performed with the SIMCA-P software (v. 13.0, Umetrics, Umeå, Sweden) with unit variance (UV) scaling methods (Kim et al., 2010).

### Results

### Jasmonate Induces the Efficacy of Antimicrobials Produced by Streptomyces MBT3

Actinomycetes are very abundantly present in the plant rhizosphere. We hypothesized that plants might very well secrete compounds that may act as elicitors of antibiotics or other beneficial metabolites produced by these actinomycetes, in order to increase fitness of the plant. To test this, 44 efficient antibiotic-producing Actinomycetes were screened for their general secondary metabolite production profiles. This revealed that Streptomyces strain MBT3 produced several plant-like compounds, including hydrocinnamates, and is a known producer of the peptide antibiotic actinomycin. This actinomycete was tested for its response to treatment with the plant defense hormone jasmonate and salicylate. For this, MBT3 was grown for 7 days in MM with mannitol and glycerol as the carbon sources and then methyl-jasmonate (100 μM) or salicylic acid (250 μM) were added to the cultures. After induction, mycelia were incubated for another 24 hours and biomass removed. The ethyl acetate extract of the culture fluid of MBT3 was analyzed further for metabolic profile and antimicrobial activity against Bacillus subtilis.

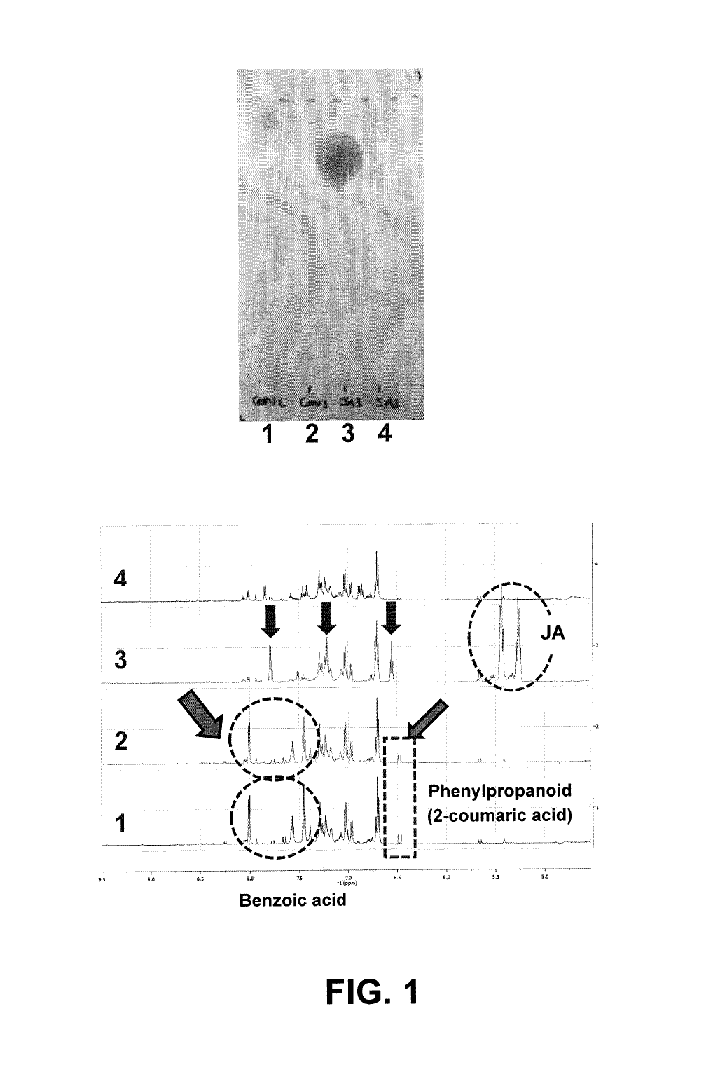

Antimicrobial activity of the extracts was analyzed using thin layer chromatography (TLC) biograms. For this, EtOAc extracts were separated on a TLC plate (FIG. 1) and subsequently tested by replication to plates overlayed with softagar (0.6% w/v agar) containing B. subtilis as the indicator strain. Surprisingly, while in control samples (addition of water or EtOAc) or samples to which salicylic acid was added there was no effect on bioactivity of MBT3, the addition of methyl jasmonate strongly enhanced the antimicrobial activity of MBT3. This suggested the induction of an antibiotic produced by MBT3.

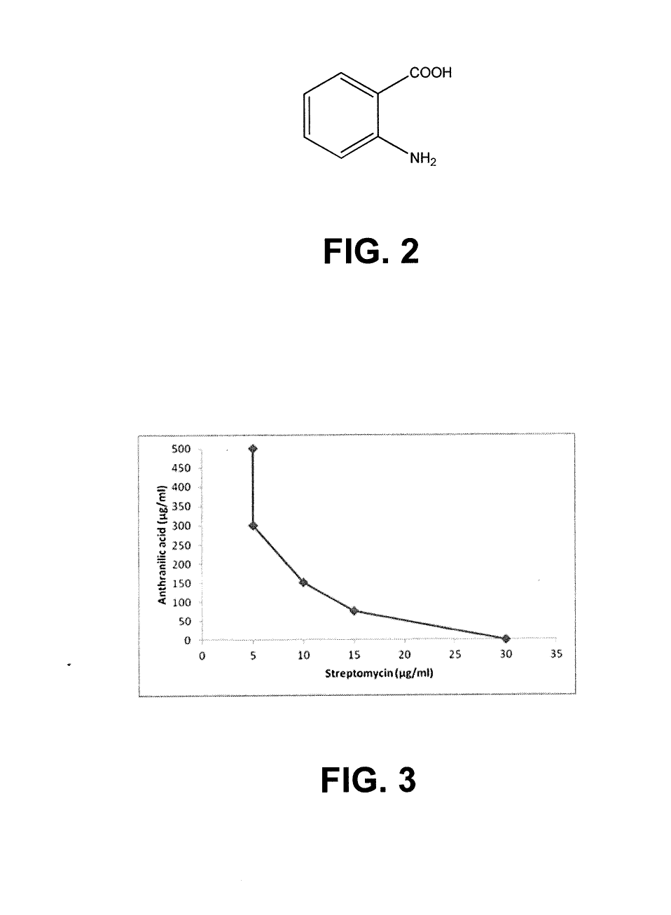

To analyze the antimicrobial activity in the active spot on the TLC plate (FIG. 1), the resin was scraped off the TLC plate and analyzed by MALDI-TOF mass spectrometry. To establish which compound was responsible for the MJ-triggered antimicrobial activity of Streptomyces MBT3 against B. subtilis, in other words was differentially produced in the presence of MJ relative to the other conditions, nuclear magnetic resonance (NMR)-based metabolic profiling was performed on the extracts. NMR analysis readily provides comprehensive structural information of both previously unknown and known metabolites, which has been applied successfully to the analysis of complex biological mixtures such as cell cultures (Anthony et al., 1996; Florian et al., 1995), and microbial cultures (Lohmeier-Vogel et al., 1995; Ramos et al., 1995). ¹H (proton) NMR spectroscopy identifies low-molecular-weight biological compounds at low concentrations and in crude culture supernatants. The profiles were, therefore, analyzed with ¹H-NMR spectroscopy (FIG. 1). This demonstrated that after treatment with methyl-jasmonate (MJ), the levels of benzoic acid and 2-coumaric acid were strongly decreased. However, a different metabolite (upper arrows in FIG. 1) was induced specifically in extracts from MJ-treated cultures. Unexpectedly, the structure of this MJ-induced compound was identified as the primary metabolite anthranilic acid on the basis of 2D NMR spectroscopy (FIG. 2).

Anthranilic acid itself has been reported for the mild growth inhibitory effect against, for example, Legionella pneumophila (Sasaki et al., 2012), acting via the inhibition of penicillin binding proteins (Sosic et al., 2012). Methyl-anthranilate is a food grade compound and is used as a repellent of birds to protect crops, such as corn, sunflowers, rice and fruits. Dimethyl anthranilate (DMA) has a similar effect. It is also used for the flavor of grape KoolAid. It is used for flavoring of candy, soft drinks (e.g., grape soda), gums, and drugs.

### Anthranilic Acid has a Synergistic Effect on Many Different Antibiotics

Anthranilate is a compound that is produced during primary metabolism and produced by many different eukaryotic and prokaryotic organisms. The effect of anthranilic acid on the bioactivity of various antibiotics was tested at different concentrations. The compound itself did not show any activity against B. subtilis at concentrations below 5 mg/ml. The MIC value of the aminoglycoside streptomycin was determined and assessed at 31.25±0.05 μg/ml. Surprisingly, addition of the metabolite anthranilate had a major effect on the susceptibility of the indicator strain B. subtilis to streptomycin, and it was decreased up to 6 times with concentration dependent manner when combined with anthranilic acid (FIG. 2). At a concentration of 75 μg/μl anthranilate, the MIC of streptomycin was reduced by 50% to 15 μg/μl, while at concentrations of 300 μg/μl and higher, a six-fold reduction in the MIC was observed.

To test how broad-spectrum the effect on the bioactivity of antibiotics was, the MICs of a wide range of different antibiotics were determined in the presence and absence of anthranilate (Tables 2 and 3). The MIC values of many different antibiotics were decreased when combined with anthranilic acid at a concentration of around 2 mM (Table 3). These antibiotics included the aminoglycosides gentamycin, kanamycin, neomycin and streptomycin, the β-lactam antibiotic penicillin G, the lincosamide-type macrolide antibiotic clindamycin and the non-ribosomal peptide (NRPS) antibiotic actinomycin. No synergistic effect was observed when anthranilate was tested together with the glycopeptide antibiotic vancomycin.

To determine the selectivity of anthranilic acid, a large range of chemically and functionally related compounds were examined for their possible synergistic effect on the bioactivity of streptomycin (Table 4). Most of the compounds did not have an effect on the MIC of streptomycin. The exceptions were catechol and 2,3-dihydroxybenzoic acid, which in fact resulted in a decrease of the bioactivity, and showed duplication of the MIC value of streptomycin.

Taken together, the data show that anthranilic acid enhances the activity of a broad range of antibiotics and this activity is specific to anthranilic acid itself, as none of the tested related compounds (even those with small structural changes as compared to anthranilic acid itself) had a positive effect on the bioactivity of streptomycin against Bacillus.

### REFERENCES

Anthony, M. L., P. C. McDowell, T. J. Gray, M. Blackmore, and J. K. Nicholson (1996). 1H NMR spectroscopic studies on the characterization of renal cell lines and identification of novel potential markers of in vitro nephrotoxicity. Biomarkers 1:35-43.
Arias, C. A., and B. E. Murray (2009). Antibiotic-resistant bugs in the 21st century—a clinical super-challenge. The New England Journal of Medicine 360:439-443.
Florian, C. L., N. E. Preece, K. K. Bhakoo, S. R. Williams, and M. D. Noble (1995). Cell type-specific fingerprinting of meningioma and meningeal cells by proton nuclear magnetic resonance spectroscopy. Cancer Res. 55:420-427.
Hopwood, D. A. (2007). Streptomyces in nature and medicine: the antibiotic makers. New York: Oxford University Press.
Jellema, R. H. (2009). Comprehensive chemometrics, chemical and biochemical data analysis. Oxford: Elsevier.
Kieser, T., M. J. Bibb, M. J. Buttner, K. F. Chater, and D. A. Hopwood (2000). Practical Streptomyces genetics. The John Innes Foundation, Norwich, United Kingdom.
Kim, H. K., Y. H. Choi, and R. Verpoorte (2010). NMR-based metabolomic analysis of plants. Nat. Protoc. 5:536-549.
Klevens, R. M., M. A. Morrison, J. Nadle, S. Petit, K. Gershman, S. Ray, L. H. Harrison, R. Lynfield, G. Dumyati, J. M. Townes, A. S. Craig, E. R. Zell, G. E. Fosheim, L. K. McDougal, R. B. Carey, and S. K. Fridkin (2007). Invasive methicillin-resistant Staphylococcus aureus infections in the United States. JAMA: The Journal of the American Medical Association 298:1763-1771.
Lohmeier-Vogel, E. M., B. Hahn-Hagerdal, and H. J. Vogel (1995). Phosphorus-31 and carbon-13 nuclear magnetic resonance study of glucose and xylose metabolism in agarose-immobilized Candida tropicalis. Appl. Environ. Microbiol. 61:1420-1425.
Ramos, A., J. S. Lolkema, W. N. Konings, and H. Santos (1995). Enzyme Basis for pH Regulation of Citrate and Pyruvate Metabolism by Leuconostoc oenos. Appl. Environ. Microbiol. 61:1303-1310.
Sambrook, J., E. F. Fritsch, and T. Maniatis (1989). Molecular Cloning: A Laboratory Manual. Cold Spring harbor, N.Y.: Cold Spring Harbor laboratory press.
Sasaki, T., S. Mizuguchi, and K. Honda (2012). Growth inhibitory effects of anthranilic acid and its derivatives against Legionella pneumophila. J. Biosci. Bioeng. 113:726-729.
Sosic, I., S. Turk, M. Sinreih, N. Trost, O. Verlaine, A. Amoroso, A. Zervosen, A. Luxen, B. Doris, and S. Gobec (2012). Exploration of the chemical space of novel naphthalene-sulfonamide and anthranilic Acid-based inhibitors of penicillin-binding proteins. Acta. Chim. Slov. 59:280-388.
WHO-Media-centre (2012) Antimicrobial resistance WHO.
Zhu, H. (2014) Environmental and Metabolomic Study of Antibiotic Production by Actinomycetes. Leiden: Leiden University.

TABLE 1
Some functional derivatives of anthranilic acid.

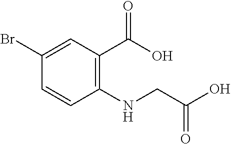 [SMILES: O=C(O)CNc1ccc(Br)cc1C(=O)O]

Carboxymethyl-anthranilic acids and derivatives thereof such as the depicted 5-bromo-
carboxymethyl-anthranilic acid. Br can be replaced by I, F, Cl.
Br, I, F, Cl can be positioned in the 3, 4, or 5 position.

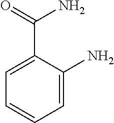

Anthranilamide

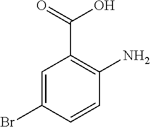 [SMILES: Nc1ccc(Br)cc1C(=O)O]

5-Bromo-anthranilic acid. Br can be replaced by I, F, Cl.
Br, I, F, Cl can be positioned in the 3, 4, or 5 position.

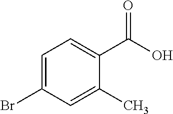

Br may be replaced by I, F, Cl. Br, I, F, Cl can be positioned in the 3, 4, or 5 position.

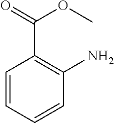

methyl anthranilate and compounds
wherein instead of the —COO—CH₃ group, a —COO—(CH₂)ₙ—CH₃ is present wherein n =
1, 2, 3, 4, or 5; or
wherein instead of the —COO—CH₃ group, a —COO—(CₓH_{y})ₙ group is present, wherein
n = 2, 3, 4, 5, or 6, and the (CₓH_{y})ₙ group comprises one or more unsaturated bonds,
wherein when the (CₓH_{y})ₙ group comprises one unsaturated bonds x = n and
y = 2n-1;
wherein when the (CₓH_{y})ₙ group comprises two unsaturated bonds, n = 4, 5 or
6; and x = n and y = 2n-3;
wherein when the (CₓH_{y})ₙ group comprises three unsaturated bonds, n = 6; and
x = 6 and y = 7.
Particularly preferred compounds are:

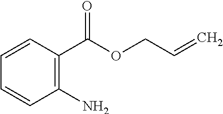

allyl anthranylate; and compounds having instead of the —COO—CH₂—CH═CH₂ group, a
—COO—(CH₂)ₙ—CH═CH₂ group wherein n = 2, 3, 4 or 5; and

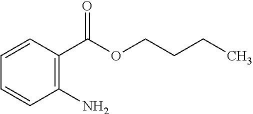

butylanthranilate

TABLE 2
MIC values of antibiotics against B. subtilis.

| Type of antibiotics | | |
|---|---|---|
| (Mechanism) | Antibiotics | MIC (ug/ml) |
| Protein synthesis | Streptomycin (sulfate) | 31.2 |
| inhibition (irreversible | Neomycin (sulfate) | 62.5 |
| binding to 30S ribosomal | Kanamycin (sulfate) | 125 |
| subunit) | Gentamycin (sulfate) | 1.25 |
| | Apramycin (sulfate) | 1.953 |
| Protein synthesis | Erythromycin | 0.122 |
| inhibition (different anti- | Chloramphenicol | 125 |
| 50S mechanisms) | Clindamycin (HCl) | 0.488 |
| Cell wall synthesis | Penicillin G (sodium salt) | 0.0062 |
| inhibition (competitive | Ampicillin (sodium salt) | 0.0153 |
| inhibition of the | Cephalexin (1H₂O) | 0.122 |
| transpeptidase enzyme) | | |
| Other cell wall inhibitor | Vancomycin (HCl) | 0.156 |
| (Disrupts peptidoglycan | | |
| cross-linkage | | |
| DNA synthesis inhibition | Nalidixic acid | 3.1 |
| RNA synthesis inhibition | Actinomycin D | 0.156 |
| (different mechanisms) | Actinomycin C₂ | 0.25 |
| | Actinomycin C | 0.078 |
| | Rifampin | 0.0122 |

TABLE 3
MIC value of antibiotics against B. subtilis
in the presence of anthranilic acid (2.2 mM)

| | | MIC with | |
|---|---|---|---|
| | MIC | anthranilic | |
| | (ug/ml) | acid (2.2 mM) | Effect |
| Streptomycin (sulfate) | 31.2 | 5 | Lower MIC |
| Neomycin (sulfate) | 62.5 | 15.6 | Lower MIC |
| Kanamycin (sulfate) | 125 | 31.2 | Lower MIC |
| Gentamycin (sulfate) | 1.25 | 0.625 | Lower MIC |
| Apramycin (sulfate) | 1.953 | 1.953 | No effect |
| Chloramphenicol | 125 | 125 | No effect |
| Clindamycin (HCl) | 0.488 | 0.244 | Lower MIC |
| Penicillin G (sodium salt) | 0.0062 | 0.0031 | Lower MIC |
| Cephalexin (1H₂O) | 0.122 | 0.061 | Lower MIC |
| Vancomycin (HCl) | 0.156 | 0.156 | No effect |
| Actinomycin D | 0.156 | 0.156 | No effect |
| Actinomycin C | 0.078 | 0.039 | Lower MIC |
| Rifampin | 0.0122 | 0.0122 | No effect |

TABLE 4
Compounds with related structure to anthranilic acid that were tested.
These compounds did not show synergistic effects on bioactivity of antibiotics.

| | | | | MIC with | |
|---|---|---|---|---|---|
| | MIC | Tested | | streptomycin | |
| Compound | (μg/ml) | concentration | | (μg/ml) | Structure |
| streptomycin | 31.2 | 31.2 | μg/ml | — | |
| Catechol | 750 | 2.7 | mM | 62.5 | 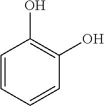 |
| 2,3-dihydroxybenzoic acid | 750 | 1.9 | mM | 62.5 | 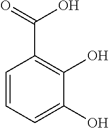 [SMILES: O=C(O)c1cccc(O)c1O] |
| Benzoic acid | >750 | 2.5 | mM | 31.2 | 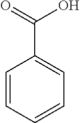 |
| Salicylic acid (sodium salt) | >1500 | 1.9 | mM | 31.2 | 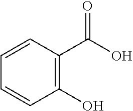 |
| L-Ascorbic acid (sodium salt) | >750 | 1.5 | mM | 31.2 | 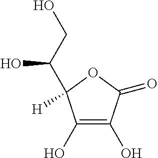 [SMILES: O=C1O[C@H]([C@@H](O)CO)C(O)=C1O] |
| L-Proline | >1500 | 0.9 | mM | 31.2 | 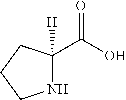 |
| D,L-Proline | >750 | 2.6 | mM | 31.2 | 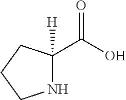 |
| 3,5-diaminobenzoic acid | >750 | 2.2 | mM | 31.2 | 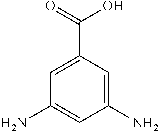 [SMILES: Nc1cc(N)cc(C(=O)O)c1] |
| 3-aminobenzoic acid | 750 | 2.2 | mM | 31.2 | 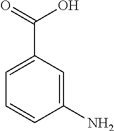 |
| 4-aminobenzoic acid | 750 | 2.2 | mM | 31.2 | 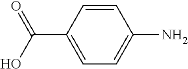 |
| Anthranilic acid amide | >1500 | 2.2 | mM | 31.2 | 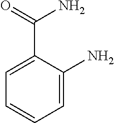 |

1. A method of treating a subject suffering from or at risk of suffering from a bacterial infection, the method comprising:
administering to the subject an antibiotic and anthranilic acid, or an antibiotic and a pharmaceutically acceptable salt, ester, hydrate, solvate or functional derivative of anthranilic acid, so as to treat the subject suffering from or at risk of suffering from a bacterial infection.

2. A method for counteracting bacterial activity and/or growth, the method comprising exposing bacteria to:
an antibiotic; and
anthranilic acid, or a pharmaceutically acceptable salt, ester, hydrate, solvate or functional derivative thereof.

3. A method for treating a subject suffering from, or at risk of suffering from, a bacterial infection, the method comprising administering to the subject:
an antibiotic; and
anthranilic acid, or a pharmaceutically acceptable salt, ester, hydrate, solvate or functional derivative thereof.

4. The method according to claim 3, wherein the subject is a human individual.

5. The method according to claim 3, wherein the individual is a child with an age below 18 years or an elderly person with an age above 50 years or an immune-compromised individual.

6. The method according to claim 3, comprising:
measuring whether a sample from the subject comprises pathogenic bacteria, proteins from pathogenic bacteria, nucleic acid from pathogenic bacteria, or antibodies against pathogenic bacteria; and
administering to the subject an antibiotic and anthranilic acid, or an antibiotic and a pharmaceutically acceptable salt, ester, hydrate, solvate or functional derivative of anthranilic acid, if the sample comprises pathogenic bacteria or proteins from pathogenic bacteria or a nucleic acid molecule from pathogenic bacteria or antibodies against pathogenic bacteria.

7. A method of increasing the antibacterial activity of an antibiotic, the method comprising:
utilizing anthranilic acid, or a pharmaceutically acceptable salt, ester, hydrate, solvate or functional derivative thereof, for increasing the antibacterial activity of an antibiotic.

8. The method according to claim 7, wherein the minimum inhibitory concentration (MIC) of the antibiotic is decreased.

9. The method according to claim 7, wherein the antibiotic is used against antibiotic-resistant bacteria.

10. The method according to claim 7, wherein an amount of the antibiotic is used that is at most half the amount required for killing bacteria in the absence of anthranilic acid or a pharmaceutically acceptable salt, ester, hydrate, solvate or functional derivative of anthranilic acid.

11. A composition or kit of parts, comprising:
anthranilic acid, or a pharmaceutically acceptable salt, ester, hydrate, solvate or functional derivative thereof; and
an antibiotic in a unit dose that is at most half the amount required for killing bacteria in the absence of anthranilic acid or a pharmaceutically acceptable salt, ester, hydrate, solvate or functional derivative of anthranilic acid.

12. The kit of parts according to claim 11, wherein the antibiotic is selected from the group consisting of antibiotics that inhibit bacterial protein synthesis, antibiotics that inhibit cell wall synthesis, antibiotics that disrupt peptidoglycan cross-linkage, antibiotics that inhibit bacterial DNA synthesis and antibiotics that inhibit bacterial RNA synthesis.

13. The kit of parts of claim 11, wherein the antibiotic is selected from the group consisting of streptomycin, neomycin, kanamycin, gentamycin, clindamycin, penicillin G, cephalexin, actinomycin and pharmaceutically acceptable salts, esters, hydrates and solvates thereof.

14. A method for culturing a micro-organism, the method comprising:
culturing the micro-organism in the presence of a plant-derived compound; and
determining whether the micro-organism produces a compound with antimicrobial activity and/or a compound that reduces the resistance of bacteria to an antibiotic.

15. The method according to claim 14, wherein the plant-derived compound is a plant hormone.

16. The method according to claim 15, wherein the plant hormone is a jasmonate.

17. The method according to claim 16, wherein the jasmonate is jasmonic acid, methyl jasmonate or jasmone.

18. The method according to claim 9, wherein the antibiotic-resistant bacteria is a multidrug-resistant bacteria.
